(19) United States
(12) Patent Application Publication
(10) Pub. No.: US 2026/0118302 A1
(43) Pub. Date: Apr. 30, 2026

(54) GAS SENSOR

(71) Applicant: TDK Corporation, Tokyo (JP)

(72) Inventors: Makoto SHIBATA, Tokyo (JP); Takumi MATSUO, Tokyo (JP); Koji HIRAI, Tokyo (JP); Yoshio KAITA, Tokyo (JP); Yoshikatsu TANAKA, Tokyo (JP); Kei TANABE, Tokyo (JP)

(21) Appl. No.: 19/345,013

(22) Filed: Sep. 30, 2025

(30) Foreign Application Priority Data
Oct. 28, 2024 (JP) 2024-188907

(51) Int. Cl. G01N 25/18; G01N 33/00

(52) U.S. Cl. CPC G01N 25/18; G01N 33/0016

(57) ABSTRACT

Disclosed herein is a gas sensor that includes first and second heaters, a first thermosensitive element that varies in temperature in accordance with a change in a temperature of the first heater, a second thermosensitive element that varies in temperature in accordance with a change in a temperature of the second heater, and a control circuit configured to calculate a concentration of a gas to be measured. The first and second heaters are heated to a first temperature zone and a second temperature zone higher than the first temperature zone, respectively, during gas concentration measurement. The temperature change directions of the first and second thermosensitive elements with respect to an increase in the concentration of the gas to be measured are opposite to each other during the gas concentration measurement.

### CROSS-REFERENCE TO RELATED APPLICATION

This application claims the benefit of Japanese Patent Application No. 2024-188907, filed on Oct. 28, 2024, the entire disclosure of which is incorporated by reference herein.

### BACKGROUND OF THE ART

### Field of the Art

The present invention relates to a gas sensor and, more particularly, to a gas sensor with high detection sensitivity.

### Description of Related Art

Japanese Patent No. 6,879,060 discloses a gas sensor capable of reducing the influence of gases other than that to be measured by performing concentration measurement of a gas to be measured at two different temperature ranges with high and low detection sensitivities.

However, in the gas sensor disclosed in Japanese Patent No. 6879060, the sensitivity direction of a thermistor in a temperature range with high detection sensitivity and the sensitivity direction of a thermistor in a temperature range with low detection sensitivity are the same, so that detection sensitivity deteriorates by a change in the characteristics of the thermistor obtained in the temperature range with low detection sensitivity.

### SUMMARY

A gas sensor according to an aspect of the present disclosure includes: a first heater; a second heater; a first thermosensitive element that varies in temperature in accordance with a change in a temperature of the first heater; a second thermosensitive element that varies in temperature in accordance with a change in a temperature of the second heater; and a control circuit, wherein the first and second thermosensitive elements are connected in series, the first and second heaters are heated to a first temperature zone and a second temperature zone higher than the first temperature zone, respectively, during gas concentration measurement, temperature change directions of the first and second thermosensitive elements with respect to an increase in a concentration of a gas to be measured are opposite to each other during the gas concentration measurement, and the control circuit is configured to calculate the concentration of the gas to be measured based on a gas detection signal appearing at a node between the first and second thermosensitive elements.

A gas sensor according to another aspect of the present disclosure includes: a first heater; a second heater; a first thermosensitive element that varies in temperature in accordance with a change in a temperature of the first heater; a second thermosensitive element that varies in temperature in accordance with a change in a temperature of the second heater; and a control circuit, wherein the first and second heaters are heated to a first temperature zone and a second temperature zone higher than the first temperature zone, respectively, during gas concentration measurement, temperature change directions of the first and second thermosensitive elements with respect to an increase in a concentration of a gas to be measured are opposite to each other during the gas concentration measurement, and the control circuit is configured to calculate the concentration of the gas to be measured based on the difference between a first output voltage caused by the first thermosensitive element and a second output voltage caused by the second thermosensitive element.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 100 according to a first embodiment of the technology described herein;

FIG. 2 is a graph for explaining a temperature-dependent change in the thermal conductivity ratio of CO₂ gas to air;

FIG. 3 is a graph for explaining an example of changes in the resistance values of the thermistors Rd1 and Rd2 in accordance with a change in the CO₂ gas concentration in the measurement atmosphere;

FIG. 4 is a timing chart for explaining the operation of the gas sensor 100;

FIG. 5 is a circuit diagram illustrating the configuration of a gas sensor 200 according to a second embodiment of the technology described herein; and

FIG. 6 is a circuit diagram illustrating the configuration of a gas sensor 300 according to a third embodiment of the technology described herein.

### DETAILED DESCRIPTION OF THE EMBODIMENTS

The present disclosure describes a gas sensor having improved detection sensitivity.

Some embodiments of the present disclosure will be explained below in detail with reference to the accompanying drawings.

### First Embodiment

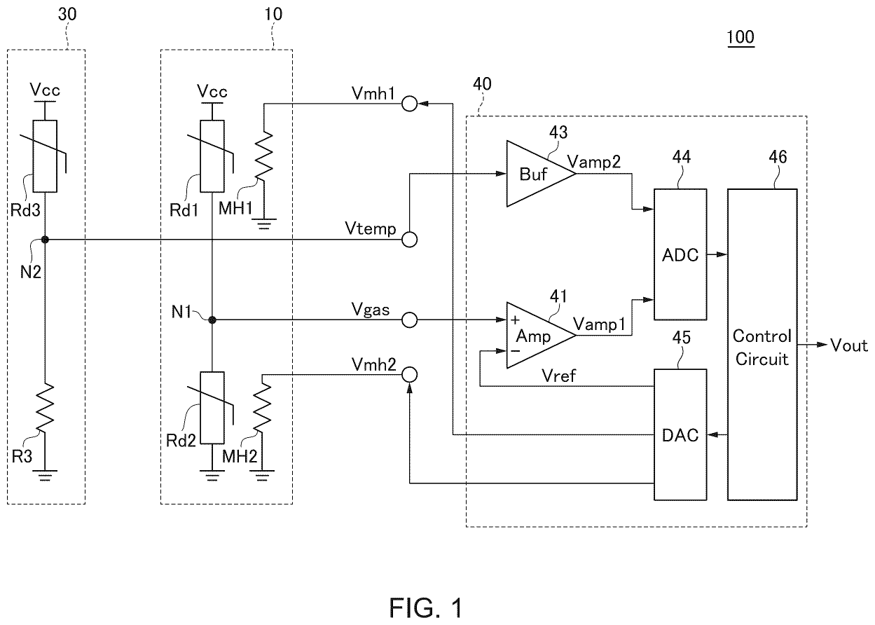

FIG. 1 is a circuit diagram illustrating the configuration of a gas sensor 100 according to a first embodiment of the technology described herein.

As illustrated in FIG. 1, the gas sensor 100 according to the first embodiment includes a sensor part 10 that generates a gas detection signal Vgas according to the concentration of a gas to be measured, a temperature sensor 30 that generates a temperature detection signal Vtemp according to an environmental temperature, and a signal processing circuit 40. Although not particularly limited, the gas sensor 100 according to the first embodiment is a heat-conduction type gas sensor for detecting the concentration of CO₂ gas in measurement atmosphere.

The sensor part 10 includes thermistors Rd1 and Rd2 connected in series in this order between a power supply Vcc and a ground GND and heaters MH1 and MH2. The thermistor Rd1 varies in temperature according to a change in the temperature of the heater MH1, and the thermistor Rd2 varies in temperature according to a change in the temperature of the heater MH2. The gas detection signal Vgas output from the sensor part 10 appears at a node N1 between the thermistors Rd1 and Rd2. The thermistors Rd1 and Rd2 are each a resistor whose resistance value varies with temperature. Examples of the material of the thermistors Rd1 and Rd2 include vanadium oxide, amorphous silicon, polycrystalline silicon, an oxide with a spinel crystal structure containing manganese, titanium oxide, and yttrium-barium-copper oxide.

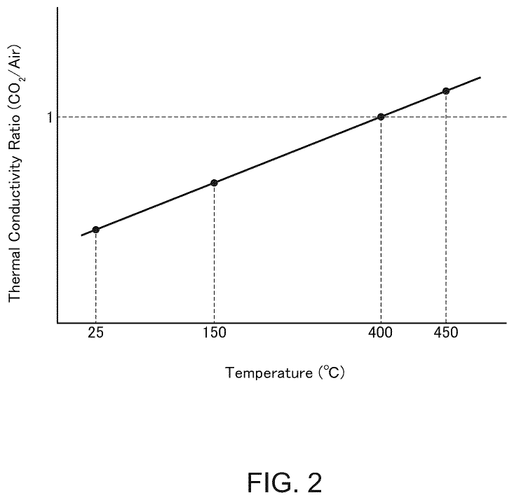

FIG. 2 is a graph for explaining a temperature-dependent change in the thermal conductivity ratio of CO₂ gas to air.

As illustrated in FIG. 2, the thermal conductivity of CO₂ gas is smaller than that of air at room temperature (25° C.) (thermal conductivity ratio<1); however, the thermal conductivity ratio increases as the temperature rises and becomes higher than the thermal conductivity of air when temperature exceeds a predetermined value (thermal conductivity ratio>1). A threshold temperature at which the relation in thermal conductivity between the CO₂ gas and air is inverted, that is, a temperature at which the thermal conductivity ratio becomes 1 is about 400° C. During gas concentration measurement operation, the heater MH1 is heated to around 150° C. (an example of a first temperature zone) which is a temperature lower than the threshold temperature, and the heater MH2 is heated to around 430° C. (an example of a second temperature zone) which is a temperature exceeding the threshold temperature. The first temperature zone as the heating temperature of the heater MH1 refers to a predetermined temperature zone included in a range of 100° C. or higher and 300° C. or lower, for example. The second temperature zone as the heating temperature of the heater MH2 refers to a predetermined temperature zone included in a range of higher than 400° C. and 450° C. or lower. The “temperature zone” in the present specification has a temperature width equal to or less than 1° C., for example. For example, a temperature zone around 150° C. may be 149.5° C. or higher and 150.5° C. or lower. Further, for example, a temperature zone around 430° C. may be 429.5° C. or higher and 430.5° C. or less. When the heater MH1 is heated to 150° C. under a situation where the thermistor Rd1 is positioned near the heater MH1, the thermistor Rd1 is also heated to about 150° C. When the heater MH2 is heated to 430° C. under a situation where the thermistor Rd2 is positioned near the heater MH2, the thermistor Rd2 is also heated to about 430° C. The thermistor Rd1 is designed to have a predetermined resistance value when being heated to, for example, 150° C., while the thermistor Rd2 is designed to have a predetermined resistance value when being heated to, for example, 430° C. Such a configuration can be achieved by adjusting the widths of a pair of electrodes provided in the thermistor Rd1 and distance therebetween and the widths of a pair of electrodes provided in the thermistor Rd2 and distance therebetween.

When CO₂ gas is present in the measurement atmosphere in a state where the heaters MH1 and MH2 are heated, the heat dissipation characteristics of the heaters MH1 and MH2 change according to the concentration of the CO₂ gas. These changes appear as changes in the temperatures of the thermistors Rd1 and Rd2, i.e., changes in the resistance values thereof.

As described using FIG. 2, the thermal conductivity of CO₂ gas is smaller than that of air (thermal conductivity ratio<1) in a temperature zone lower than about 400° C. as the threshold temperature, so that when CO₂ gas is present in the measurement atmosphere in a state where a certain power is applied to the heater MH1 to heat the same, the temperature of the heater MH1 rises as the concentration of CO₂ gas becomes high with the result that the temperature of the thermistor Rd1 also rises. Therefore, assume that heating is performed such that the temperature of the heater MH1 becomes 150° C. when the concentration of CO₂ gas in the measurement atmosphere indicates a concentration value (e.g., 400 ppm) of CO₂ gas under ordinary atmospheric environment. In this case, when the concentration of CO₂ gas present in the measurement atmosphere exceeds the concentration value under ordinary atmospheric environment, the temperature of the heater MH1 rises in accordance with the concentration of CO₂ gas and exceeds 150° C., and thus the temperature of the thermistor Rd1 also becomes higher than that in the case where the concentration of CO₂ gas in the measurement atmosphere indicates a concentration value (e.g., 400 ppm) of CO₂ gas under ordinary atmospheric environment. As a result, when, for example, the thermistor Rd1 has a negative resistance temperature coefficient (when the thermistor Rd1 is an NTC thermistor), the resistance value of the thermistor Rd1 lowers as the CO₂ gas concentration in the measurement atmosphere increases.

To the contrary, the thermal conductivity of CO₂ gas is larger than that of air (thermal conductivity ratio>1) in a temperature zone higher than about 400° C. as the threshold temperature, so that when CO₂ gas is present in the measurement atmosphere in a state where a certain power is applied to the heater MH2 to heat the same, the temperature of the heater MH2 lowers as the concentration of CO₂ gas increases, with the result that the temperature of the thermistor Rd2 also lowers. Therefore, assume that heating is performed such that the temperature of the heater MH2 becomes 430° C. when the concentration of CO₂ gas in the measurement atmosphere indicates a concentration value (e.g., 400 ppm) of CO₂ gas under ordinary atmospheric environment. In this case, when the concentration of CO₂ gas present in the measurement atmosphere exceeds the concentration value under ordinary atmospheric environment, the temperature of the heater MH2 lowers in accordance with the concentration of CO₂ gas and falls below 430° C., and thus the temperature of the thermistor Rd2 also becomes lower than that in the case where the concentration of CO₂ gas in the measurement atmosphere indicates a concentration value (e.g., 400 ppm) of CO₂ gas under ordinary atmospheric environment. As a result, when, for example, the thermistor Rd2 has a negative resistance temperature coefficient (when the thermistor Rd2 is an NTC thermistor), the resistance value of the thermistor Rd2 increases as the CO₂ gas concentration in the measurement atmosphere increases.

Since the thermistors Rd1 and Rd2 are connected in series between the power supply Vcc and the ground GND, the higher the CO₂ gas concentration in the measurement atmosphere is, the higher the level of the gas detection signal Vgas appearing at the node N1 becomes.

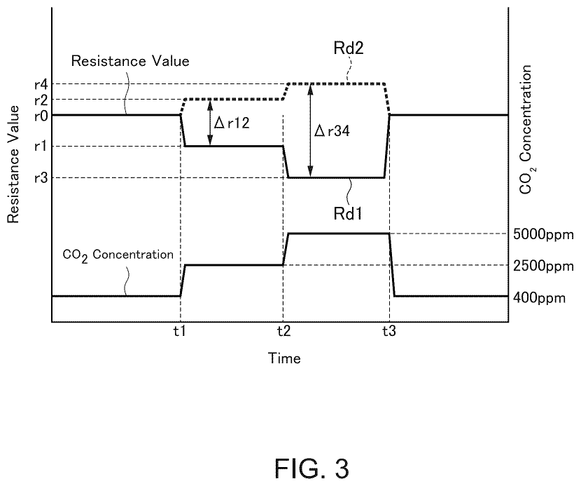

FIG. 3 is a graph for explaining an example of changes in the resistance values of the thermistors Rd1 and Rd2 in accordance with a change in the CO₂ gas concentration in the measurement atmosphere.

In the example illustrated in FIG. 3, the CO₂ gas concentration in the measurement atmosphere is 400 ppm in a period before time t1, 2500 ppm in a period from time t1 to time t2, 5000 ppm in a period from time t2 to time t3, and 400 ppm in a period after time t3. Here assume that, in the period before time t1, the resistance value (resistance value between the electrode pair provided in the thermistor Rd1) of the thermistor Rd1 and the resistance value (resistance value between the electrode pair provided in the thermistor Rd2) of the thermistor Rd2 are both r0. In this case, in the period from time t1 to time t2, the resistance value of the thermistor Rd1 lowers to r1 (<r0), whereas the resistance value of the thermistor Rd2 increases to r2 (>r0). As a result, the level of the gas detection signal Vgas appearing at the node N1 increases to a level corresponding to the ratio between the resistance values r1 and r2. For example, Vgas=Vcc/(1+r1/r2) is satisfied. In this case, the difference between the resistance values r1 and r2 is Δr12. In the period from time t2 to time t3, the resistance value of the thermistor Rd1 further lowers to r3 (<r1), whereas the resistance value of the thermistor Rd2 further increases to r4 (>r2). As a result, the level of the gas detection signal Vgas appearing at the node N1 increases to a level corresponding to the ratio between the resistance values r3 and r4. For example, Vgas=Vcc/(1+r3/r4) is satisfied. In this case, the difference between the resistance values r3 and r4 is Δr34 which is larger than the difference Δr12. The ratio r3/r4 between the resistance values r3 and r4 is smaller than the ratio r1/r2 between the resistance values r1 and r2.

As described above, during the gas concentration measurement, the temperature change directions of the thermistors Rd1 and Rd2 with respect to an increase in the CO₂ gas concentration are opposite to each other with the result that the resistance value change directions of the thermistors Rd1 and Rd2 with respect to an increase in the CO₂ gas concentration are opposite to each other. Thus, as compared to when the resistance value change directions of the thermistors Rd1 and Rd2 are the same as each other, a change in the ratio between the resistance values of the thermistors Rd1 and Rd2 to an increase in the CO₂ gas concentration becomes large. As a result, a change in the level of the gas detection signal Vgas appearing at the node N1 with respect to an increase in the CO₂ gas concentration is further enlarged.

However, the resistance values of the thermistors Rd1 and Rd2 when the CO₂ gas concentration in the measurement atmosphere indicates a concentration value (e.g., 400 ppm) of CO₂ gas under ordinary atmospheric environment need not be the same (=r0) but may differ from each other. When the resistance values of the thermistors Rd1 and Rd2 are the same under the condition that the CO₂ gas concentration in the measurement atmosphere indicates a concentration value (e.g., 400 ppm) of CO₂ gas under ordinary atmospheric environment, the level of the gas detection signal Vgas becomes Vcc/2.

On the other hand, even when there is contained, in the measurement atmosphere, another gas that brings about no significant difference between the heat dissipation characteristics of the heater MH1 exhibited when it is heated to around 150° C. and those of the heater MH2 exhibited when it is heated to around 430° C., the concentration of this gas has little influence on the level of the gas detection signal Vgas. This allows the sensor part 10 to selectively detect the CO₂ gas concentration.

The temperature sensor 30 includes a thermistor Rd3 and a fixed resistor R3 which are connected in series between the power supply Vcc and the ground GND. The temperature detection signal Vtemp output from the temperature sensor 30 appears at a node N2 between the thermistor Rd3 and the fixed resistor R3. The temperature sensor 30 detects an environmental temperature. The environmental temperature is a temperature in the measurement atmosphere. The temperature sensor 30 may be designed so as not to be affected or so as to be hardly affected by heating by, for example, the heaters MH1 and MH2.

The signal processing circuit 40 includes a differential amplifier 41, a buffer 43, an AD converter (ADC) 44, a DA converter (DAC) 45, and a control circuit 46.

The differential amplifier 41 compares the gas detection signal Vgas and a reference potential Vref to generate an amplified signal Vamp1 which is a signal obtained by amplifying the difference in level (=Vgas−Vref) between the gas detection signal Vgas and the reference potential Vref. The buffer 43 buffers the temperature detection signal Vtemp to generate an amplified signal Vamp2. The amplified signals Vamp1 and Vamp2 are input to the AD converter 44. The AD converter 44 AD converts the amplified signals Vamp1 and Vamp2 to generate digital values and supplies them to the control circuit 46.

The control circuit 46 calculates the concentration of CO₂ gas which is a gas to be detected based on the A/D converted amplified signal Vamp1 and generates an output signal Vout indicating the CO₂ gas concentration. The control circuit 46 may calculate the CO₂ gas concentration using a calculation formula set therein. Further, the control circuit 46 supplies digital values of various control parameters to the DA converter 45. The DA converter 45 D/A converts the digital values of the various control parameters to generate heater voltages Vmh1 and Vmh2 and the reference potential Vref. The heater voltages Vmh1 and Vmh2 are applied to the heaters MH1 and MH2, respectively, whereby the heaters MH1 and MH2 are heated. The reference potential Vref is supplied to the differential amplifier 41.

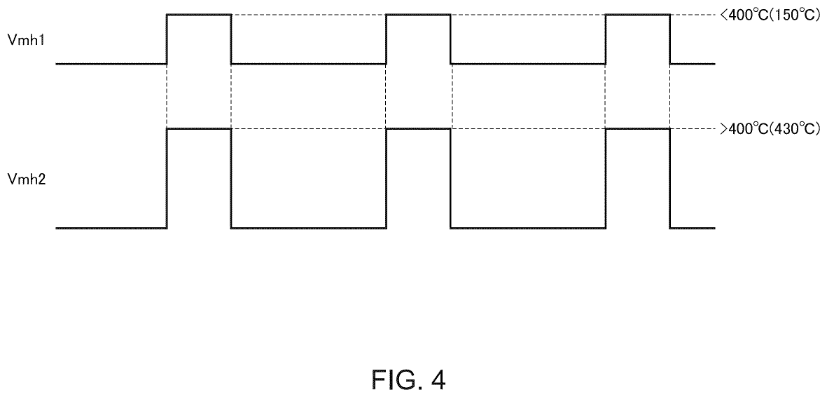

FIG. 4 is a timing chart for explaining the operation of the gas sensor 100.

As illustrated in FIG. 4, the gas sensor 100 according to the present embodiment applies the heater voltages Vmh1 and Vmh2 during the gas concentration measurement to heat the heaters MH1 and MH2 simultaneously. The heating temperature of the heater MH1 by the application of the heater voltage Vmh1 is a temperature (e.g., 150° C.) lower than the threshold temperature. The heating temperature of the heater MH2 by the application of the heater voltage Vmh2 is a temperature (e.g., 430° C.) exceeding the threshold temperature. The levels of the heater voltages Vmh1 and Vmh2 are controlled by reference to the amplified signal Vamp 2 obtained by amplifying the temperature signal Vtemp such that the temperatures of the heaters MH1 and MH2 each become a predetermined value irrespective of the environmental temperature. For example, when the concentration of CO₂ gas in the measurement atmosphere indicates a concentration value (e.g., 400 ppm) of CO₂ gas under ordinary atmospheric environment, the level of the heater voltage Vmh1 is set such that the heating temperature of the heater MH1 becomes, for example, 150° C., and the level of the heater voltage Vmh2 is set such that the heating temperature of the heater MH2 becomes, for example, 430° C. The heating temperature of the heater MH1 is not particularly limited as long as it is lower than the threshold temperature; however, as described using FIG. 2, the heat conductivity ratio approaches 1 with a rise of temperature, so that it is possible to achieve high detection sensitivity by setting the heating temperature of the heater MH1 equal to or lower than 300° C.

Then, in a state where the heaters MH1 and MH2 are heated simultaneously, the gas detection signal Vgas is taken in the signal processing circuit 40, and the output signal Vout is calculated based on the level of the Vgas and externally output. By periodically executing the above-described gas concentration measurement, the CO₂ gas concentration in the measurement atmosphere can be detected periodically.

As described above, in the gas sensor 100 according to the present embodiment, the temperature change directions of the thermistors Rd1 and Rd2 with respect to an increase in the CO₂ gas concentration are opposite to each other during the gas concentration measurement with the result that the resistance value change directions of the thermistors Rd1 and Rd2 with respect to an increase in the CO₂ gas concentration are opposite to each other during the gas concentration measurement. This enlarges a change in the level of the gas detection signal Vgas in accordance with a change in the CO₂ gas concentration, making it possible to further improve detection sensitivity for the CO₂ gas concentration.

### Second Embodiment

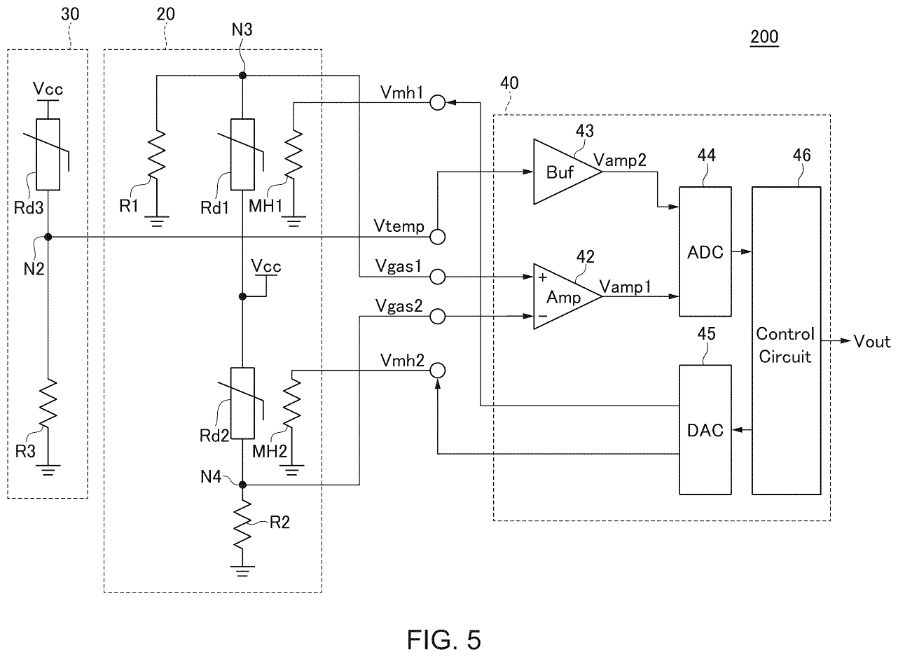

FIG. 5 is a circuit diagram illustrating the configuration of a gas sensor 200 according to a second embodiment of the technology described herein.

As illustrated in FIG. 5, the gas sensor 200 according to the second embodiment differs from the gas sensor 100 according to the first embodiment in that the sensor part 10 is replaced with a sensor part 20 and that the differential amplifier 41 included in the signal processing circuit 40 is replaced with a differential amplifier 42. Other basic configurations are the same as those of the gas sensor 100 according to the first embodiment, so the same reference numerals are given to the same elements, and overlapping description will be omitted.

The sensor part 20 includes the thermistor Rd1 and a fixed resistor R1 connected in series in this order between the power supply Vcc and the ground GND, the thermistor Rd2 and a fixed resistor R2 connected in series in this order between the power supply Vcc and the ground GND, and the heaters MH1 and MH2. The thermistor Rd1 varies in temperature according to a change in the temperature of the heater MH1, and the thermistor Rd2 varies in temperature according to a change in the temperature of the heater MH2. A gas detection signal Vgas1 appears at a node N3 between the thermistor Rd1 and the fixed resistor R1, and a gas detection signal Vgas2 appears at a node N4 between the thermistor Rd2 and the fixed resistor R2. The resistance values of the fixed resistors R1 and R2 may be close to the resistance values of the thermistors Rd1 and Rd2, respectively, in the gas concentration measurement when the CO₂ gas concentration in the measurement atmosphere indicates a concentration value (e.g., 400 ppm) of CO₂ gas under ordinary atmospheric environment.

The differential amplifier 42 included in the signal processing circuit 40 compares the gas detection signals Vgas1 and Vgas2 to generate an amplified signal Vamp1 which is a signal obtained by amplifying the level difference (=Vgas1−Vgas2) between the gas detection signals Vgas1 and Vgas2.

Description will be made referring back to FIG. 3. Assume that, in the period before time t1, the resistance values of the thermistors Rd1 and Rd2 are both r0. In this case, in the period from time t1 to time t2, the resistance value of the thermistor Rd1 lowers to r1 (<r0), whereas the resistance value of the thermistor Rd2 increases to r2 (>r0). As a result, the level of the gas detection signal Vgas1 appearing at the node N3 increases, whereas the level of the gas detection signal Vgas2 appearing at the node N4 lowers. The difference in level between the gas detection signals Vgas1 and Vgas2 corresponds to the difference Δr12 between resistance values r1 and r2. In the period from time t2 to time t3, the resistance value of the thermistor Rd1 lowers to r3 (<r1), whereas the resistance value of the thermistor Rd2 increases to r4 (>r2). As a result, the level of the gas detection signal Vgas1 appearing at the node N3 increases, whereas the level of the gas detection signal Vgas2 appearing at the node N4 lowers. The difference in level between the gas detection signals Vgas1 and Vgas2 corresponds to the difference Δr34 between resistance values r3 and r4.

As described above, during the gas concentration measurement, the temperature change directions of the thermistors Rd1 and Rd2 with respect to an increase in the CO₂ gas concentration are opposite to each other with the result that the resistance value change directions of the thermistors Rd1 and Rd2 with respect to an increase in the CO₂ gas concentration are opposite to each other. Thus, as compared to when the resistance value change directions of the thermistors Rd1 and Rd2 are the same as each other, the difference Δr12 and difference Δr34 are enlarged. As a result, a change in the level difference (=Vgas1−Vgas2) between the gas detection signals Vgas1 and Vgas2 with respect to an increase in the CO₂ gas concentration is further enlarged.

As exemplified by the gas sensor 200 according to the second embodiment, the thermistors Rd1 and Rd2 need not necessarily be connected in series between the power supply Vcc and the ground GND, but may be connected in parallel between the power supply Vcc and the ground GND so as to calculate the concentration of a gas to be measured based on the difference between an output voltage (gas detection signal Vgas1) caused by the thermistor Rd1 and an output voltage (gas detection signal Vgas2) caused by the thermistor Rd2.

As described above, in the gas sensor 200 according to the present embodiment, the temperature change directions of the thermistors Rd1 and Rd2 with respect to an increase in the CO₂ gas concentration are opposite to each other during the gas concentration measurement, with the result that the resistance value change directions of the thermistors Rd1 and Rd2 with respect to an increase in the CO₂ gas concentration are opposite to each other during the gas concentration measurement. This enlarges a change in the level difference (=Vgas1−Vgas2) between the gas detection signals Vgas1 and Vgas2 in accordance with a change in the CO₂ gas concentration, making it possible to further improve detection sensitivity for the CO₂ gas concentration.

### Third Embodiment

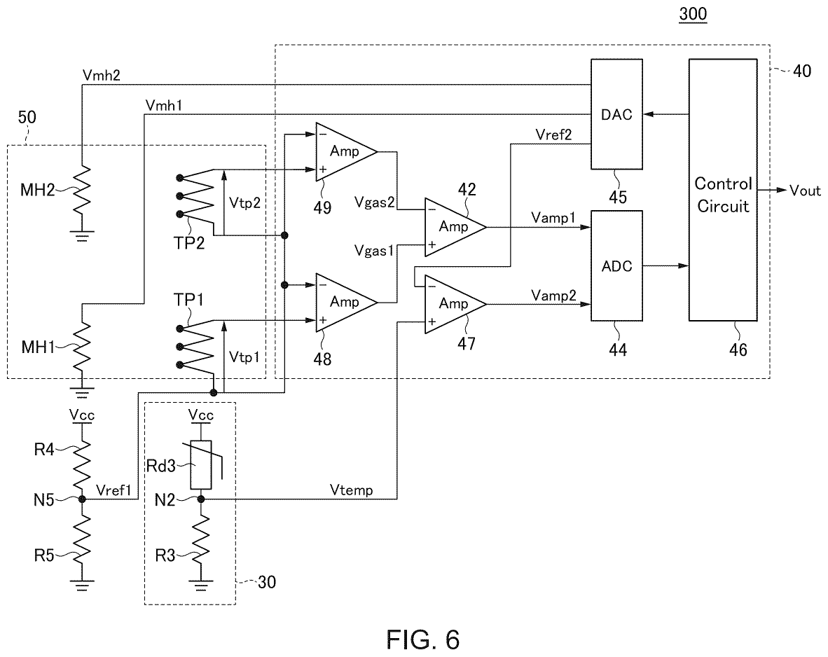

FIG. 6 is a circuit diagram illustrating the configuration of a gas sensor 300 according to a third embodiment of the technology described herein.

As illustrated in FIG. 6, the gas sensor 300 according to the third embodiment differs from the gas sensor 200 according to the second embodiment in that the sensor part 20 is replaced with a sensor part 50 and that differential amplifiers 47 to 49 are provided in the signal processing circuit 40. Other basic configurations are the same as those of the gas sensor 200 according to the second embodiment, so the same reference numerals are given to the same elements, and overlapping description will be omitted.

The sensor part 50 includes thermopile elements TP1 and TP2 and the heaters MH1 and MH2. The hot junction of the thermopile element TP1 varies in temperature in accordance with a change in the temperature of the heater MH1, and the hot junction of the thermopile element TP2 varies in temperature in accordance with a change in the temperature of the heater MH2. The thermopile elements TP1 and TP2 each vary with temperature in potential difference appearing between both ends thereof. The potential difference appearing between the both ends of the thermopile element TP1 is used as an output signal Vtp1, and the potential difference appearing between the both ends of the thermopile element TP2 is used as an output signal Vtp2. A reference potential Vref1 is generated by the fixed resistors R4 and R5. The fixed resistors R4 and R5 are connected in series between the power supply Vcc and the ground GND, and the reference potential Vref1 appears at a node N5 between the fixed resistors R4 and R5. The reference potential Vref1 and output signals Vtp1 and Vtp2 are supplied to the signal processing circuit 40. A potential supplied to the non-inverting input terminal (+) of the differential amplifier 48 has a level obtained by superimposing, on the reference potential Vref1, the output signal Vtp1 corresponding to the electromotive force of the thermopile element TP1 according to temperature. A potential supplied to the non-inverting input terminal (+) of the differential amplifier 49 has a level obtained by superimposing, on the reference potential Vref1, the output signal Vtp2 corresponding to the electromotive force of the thermopile element TP2 according to temperature.

The output signal Vtp1 is amplified by the differential amplifier 48 included in the signal processing circuit 40 to generate the gas detection signal Vgas1. The differential amplifier 48 compares the level of the reference potential Vref1 supplied to the inverting input terminal (−) thereof and the level of Vref1+Vtp1 supplied to the non-inverting input terminal (+) thereof and amplifies the difference (=Vtp1) therebetween to generate the gas detection signal Vgas1.

The output signal Vtp2 is amplified by the differential amplifier 49 included in the signal processing circuit 40 to generate the gas detection signal Vgas2. The differential amplifier 49 compares the level of the reference potential Vref1 supplied to the inverting input terminal (−) thereof and the level of Vref1+Vtp2 supplied to the non-inverting input terminal (+) thereof and amplifies the difference (=Vtp2) therebetween to generate the gas detection signal Vgas2.

As in the second embodiment, the differential amplifier 42 compares the gas detection signals Vgas1 and Vgas2 to generate the amplified signal Vamp1 which is a signal obtained by amplifying the level difference (=Vgas1−Vgas2) between the gas detection signals Vgas1 and Vgas2.

In the present embodiment, the temperature detection signal Vtemp output from the temperature sensor 30 is supplied to the differential amplifier 47 included in the signal processing circuit 40. The differential amplifier 47 compares the temperature detection signal Vtemp and the reference potential Vref2 to generate the amplified signal Vamp2 which is a signal obtained by amplifying the level difference (=Vtemp−Vref2) between the temperature detection signal Vtemp and the reference potential Vref2. Alternatively, as is the case with the gas sensor 100 according to the first embodiment and the gas sensor 200 according to the second embodiment, the amplified signal Vamp2 may be generated by buffering the temperature detection signal Vtemp using the buffer 43.

Even with such a circuit configuration, when the CO₂ gas concentration in the measurement atmosphere increases, the temperature at the hot junction of the thermopile element TP1 during the gas concentration measurement increases, so that the level of the gas detection signal Vgas1 corresponding to the thermal electromotive force of the thermopile element TP1 increases, whereas the temperature at the hot junction of the thermopile element TP2 during the gas concentration measurement lowers, so that the level of the gas detection signal Vgas2 corresponding to the thermal electromotive force of the thermopile element TP2 lowers. The temperature change directions of the thermopile elements TP1 and TP2 at their hot junctions with respect to an increase in the CO₂ gas concentration are thus opposite to each other, so that, as compared to when the temperature change directions of the thermopile elements TP1 and TP2 at their hot junctions are the same as each other, a change in the level difference (=Vgas1−Vgas2) between the gas detection signals Vgas1 and Vgas2 with respect to an increase in the CO₂ gas concentration is enlarged.

As exemplified by the gas sensor 300 according to the third embodiment, it is not essential that the thermistor is used as a thermosensitive element, but another type of thermosensitive element, such as the thermopile element, may be used. Further, as exemplified by the gas sensor 300 according to the third embodiment, the concentration of a gas to be measured may be calculated based on the difference between the output voltage (gas detection signal Vgas1) caused by the thermopile element TP1 and the output voltage (gas detection signal Vgas2) caused by the thermopile element TP2.

As described above, in the gas sensor 300 according to the present embodiment, the temperature change directions of the thermopile elements TP1 and TP2 at their hot junctions with respect to an increase in the CO₂ gas concentration are opposite to each other, with the result that the change direction of the level of the thermal electromotive force of the thermopile element TP1 with respect to an increase in the CO₂ gas concentration and that of the level of the thermal electromotive force of the thermopile element TP2 with respect to an increase in the CO₂ gas concentration are opposite to each other during the gas concentration measurement. This enlarges a change in the level difference (=Vgas1−Vgas2) between the gas detection signals Vgas1 and Vgas2 in accordance with a change in the CO₂ gas concentration, making it possible to further improve detection sensitivity for the CO₂ gas concentration.

While some embodiments of the technology according to the present disclosure have been described, the technology according to the present disclosure is not limited to the above embodiments, and various modifications may be made within the scope of the present disclosure, and all such modifications are included in the technology according to the present disclosure.

The technology according to the present disclosure includes the following configuration examples, but not limited thereto.

A gas sensor according to an aspect of the present disclosure includes: a first heater; a second heater; a first thermosensitive element that varies in temperature in accordance with a change in a temperature of the first heater; a second thermosensitive element that varies in temperature in accordance with a change in a temperature of the second heater; and a control circuit, wherein the first and second thermosensitive elements are connected in series, the first and second heaters are heated to a first temperature zone and a second temperature zone higher than the first temperature zone, respectively, during gas concentration measurement, temperature change directions of the first and second thermosensitive elements with respect to an increase in a concentration of a gas to be measured are opposite to each other during the gas concentration measurement, and the control circuit is configured to calculate the concentration of the gas to be measured based on a gas detection signal appearing at a node between the first and second thermosensitive elements. With this configuration, it is possible to achieve high detection sensitivity for the gas to be measured. Further, the gas detection signal can be obtained by a half-bridge circuit including the first and second thermosensitive elements.

A gas sensor according to another aspect of the present disclosure includes: a first heater; a second heater; a first thermosensitive element that varies in temperature in accordance with a change in a temperature of the first heater; a second thermosensitive element that varies in temperature in accordance with a change in a temperature of the second heater; and a control circuit, wherein the first and second heaters are heated to a first temperature zone and a second temperature zone higher than the first temperature zone, respectively, during gas concentration measurement, temperature change directions of the first and second thermosensitive elements with respect to an increase in a concentration of a gas to be measured are opposite to each other during the gas concentration measurement, and the control circuit is configured to calculate the concentration of the gas to be measured based on the difference between a first output voltage caused by the first thermosensitive element and a second output voltage caused by the second thermosensitive element. With this configuration, it is possible to achieve high detection sensitivity for the gas to be measured. Further, the level of the second output voltage can be adjusted irrespective of the first thermosensitive element, and the level of the first output voltage can be adjusted irrespective of the second thermosensitive element.

In the above gas sensor, the gas to be measured may be CO₂ gas, the first temperature zone may be a predetermined temperature zone equal to or lower than 300° C., and the second temperature zone may be a predetermined temperature zone exceeding 400° C. This allows the CO₂ gas concentration in measurement atmosphere to be detected with high sensitivity.

What is claimed is:

1. A gas sensor comprising:
a first heater;
a second heater;
a first thermosensitive element that varies in temperature in accordance with a change in a temperature of the first heater;
a second thermosensitive element that varies in temperature in accordance with a change in a temperature of the second heater; and
a control circuit,
wherein the first and second thermosensitive elements are connected in series,
wherein the first and second heaters are heated to a first temperature zone and a second temperature zone higher than the first temperature zone, respectively, during gas concentration measurement,
wherein temperature change directions of the first and second thermosensitive elements with respect to an increase in a concentration of a gas to be measured are opposite to each other during the gas concentration measurement, and
wherein the control circuit is configured to calculate the concentration of the gas to be measured based on a gas detection signal appearing at a node between the first and second thermosensitive elements.

2. The gas sensor as claimed in claim 1,
wherein the gas to be measured is CO₂ gas,
wherein the first temperature zone is a predetermined temperature zone equal to or lower than 300° C., and
wherein the second temperature zone is a predetermined temperature zone exceeding 400° C.

3. A gas sensor comprising:
a first heater;
a second heater;
a first thermosensitive element that varies in temperature in accordance with a change in a temperature of the first heater;
a second thermosensitive element that varies in temperature in accordance with a change in a temperature of the second heater; and
a control circuit,
wherein the first and second heaters are heated to a first temperature zone and a second temperature zone higher than the first temperature zone, respectively, during gas concentration measurement,
wherein temperature change directions of the first and second thermosensitive elements with respect to an increase in a concentration of a gas to be measured are opposite to each other during the gas concentration measurement, and
wherein the control circuit is configured to calculate the concentration of the gas to be measured based on a difference between a first output voltage caused by the first thermosensitive element and a second output voltage caused by the second thermosensitive element.

4. The gas sensor as claimed in claim 3,
wherein the gas to be measured is CO₂ gas,
wherein the first temperature zone is a predetermined temperature zone equal to or lower than 300° C., and
wherein the second temperature zone is a predetermined temperature zone exceeding 400° C.
